(19) United States
(12) Patent Application Publication
(10) Pub. No.: US 2026/0141519 A1
(43) Pub. Date: May 21, 2026

(54) PROCESSING SYSTEM AND METHOD FOR MEDICAL IMAGE

(71) Applicant: IntroMedic Co., Ltd., Seoul (KR)

(72) Inventors: You Jin KIM, Muan-gun, Jeollanam-do (KR); Hong Young JEONG, Incheon (KR)

(73) Assignee: IntroMedic Co., Ltd., Seoul (KR)

(21) Appl. No.: 18/698,390

(22) PCT Filed: Aug. 2, 2023
(86) PCT No.: PCT/KR2023/011335

§ 371 Date: Apr. 4, 2024

(30) Foreign Application Priority Data
Jul. 27, 2023 (KR) 10-2023-0098094

(51) Int. Cl. G06T 7/00; A61B 1/00; A61B 1/04; G06T 7/90; G06V 10/54; G06V 10/56; G06V 10/74; G06V 10/764; G06V 10/776; G06V 10/82

(52) U.S. Cl. CPC G06T 7/0014; A61B 1/000096; A61B 1/00016; A61B 1/041; G06T 7/90; G06V 10/761; G06V 10/764; G06V 10/82; G06T 2207/10016; G06T 2207/10024; G06T 2207/10068; G06T 2207/20081; G06T 2207/20084; G06T 2207/20092; G06T 2207/30096; G06T 2207/30101; G06V 10/54; G06V 10/56; G06V 10/776; G06V 2201/03

(57) ABSTRACT

A medical image processing system according to the present invention includes a communication unit configured to receive images captured from a capsule endoscope; and a processing unit configured to generate readout images from the captured images using lesion model results trained by a lesion classification method, wherein the processing unit configured to apply the captured images to a pre-trained abnormal lesion classification model to classify images containing pre-set lesion, reclassify the classified images into lesion images and normal images, select representative images representing the lesion images from the classified lesion images, and generate the selected representative images as the readout images.

### BACKGROUND OF THE DISCLOSURE

### Technical Field

The present invention relates to a processing system and method for medical images, and more particularly to a processing system and method for medical images provided by a capsule endoscope.

### Background Art

In general, capsule endoscopes are used for the diagnosis of various intestinal diseases. In the diagnosis of intestinal diseases, lesions are diagnosed based on in-vivo medical images acquired by a capsule endoscope inserted into the human body.

A conventional capsule endoscope is already disclosed by “Korean Patent Publication No. 10-1969982 (Capsule endoscope device, magnetic controller and capsule endoscope system, Apr. 11, 2019)”. The disclosed invention enables medical images to be acquired through a capsule endoscope inserted into the human body, and diagnosis of lesions based on the medical images.

In the diagnosis of such lesions, an abnormal lesion is detected from the medical image and a readout image is generated. Then, a clinician diagnoses the lesion through the readout image. In the process of diagnosing the lesion, the clinician reviews a large number of video frames included in the readout image. In particular, the time it takes for a specialist to read a lesion from video frames is known to take anywhere from 30 minutes to more than two hours, depending on their skill level. In other words, lesion diagnosis takes time to generate the readout images and time to read the lesions. Thus, there has been a problem that lesion diagnosis takes a long time.

Korean Patent Publication No. 10-1969982 (Capsule endoscopy device, magnetic controller, and capsule endoscopy system, Apr. 11, 2019)

### SUMMARY OF THE DISCLOSURE

It is an object of the present invention to provide a medical image processing system and processing method that reduces time when generating a readout image and reduces the number of video frames of the readout image provided to a clinician.

A medical image processing system according to the present invention includes a communication unit configured to receive images captured from a capsule endoscope; and a processing unit configured to generate readout images from the captured images using lesion model results trained by a lesion classification method, wherein the processing unit configured to apply the captured images to a pre-trained abnormal lesion classification model to classify images containing pre-set lesion, reclassify the classified images into lesion images and normal images, select representative images representing the lesion images from the classified lesion images, and generate the selected representative images as the readout images.

The abnormal lesion classification model includes a YOLO (You Only Look Once) series model.

The abnormal lesion classification model includes at least one of YOLO v4 and YOLO v8 models.

In labeling of training data for the abnormal lesion classification model, an expert identifies a lesion present in the training data, and performs labeling by applying the text for the lesion and a preset marking method to the lesion present in the training data.

In marking of the lesion, the lesion is marked with a rectangle or a polygon surrounding the lesion

The captured images may include at least one of hemorrhagic, inflammatory, vascular, and polypoid lesion images, and the abnormal lesion classification model may binary classify the captured images into the lesion images and the normal images regardless of a type of the lesion in the reclassification process.

The abnormal lesion classification model applies a bounding box to a lesion included in the lesion image.

The abnormal lesion classification model may statistically calculate a threshold for each lesion comprising at least one of the hemorrhagic, inflammatory, vascular, and polypoid lesions, and when the captured images is provided, may binary classifies the lesion images and the normal images based on the threshold for each lesion.

In the selection of the representative images, a pre-trained video frame processing model may be applied, and the video frame processing model may extract features of color and texture of the lesion images to compare the similarity between images.

In the similarity analysis, the similarity may be analyzed based on Bhattacharyya Distance.

In the similarity analysis, the lesion images being compared with each other may be HSV transformed, and histograms of color values and saturation values may be calculated by adjusting the number of bins of color and saturation values.

In the selection of the representative images, comparisons between a reference image and subsequent images may be sequentially performed, when a subsequent image with a different similarity from the reference image appears, a comparison between the reference image and the subsequent image may be sequentially performed using the subsequent image with the different similarity as the reference image, and the reference images may be selected as the representative images.

In the selection of the representative images, the representative images may be selected so that the number of images between a pair of representative images does not exceed n.

In the selection of the representative images, comparisons between a reference image and subsequent images may be sequentially performed, a nth subsequent image after the reference image may be selected as a reference image, and the reference image may be selected as a representative image.

A method of processing medical images according to the present invention includes the steps of receiving images captured from a capsule endoscope, reclassifying the captured images into lesion images and normal images after applying the captured images to a pre-trained abnormal lesion classification model to classify images containing pre-set lesions, selecting representative images that represent the lesion images from the classified lesion images, and generating the selected representative images as readout images.

The medical image processing system and processing method according to the present invention have the effect of reducing the time for reading medical images, improving the reading efficiency of clinicians, and reducing the waiting time for patients.

The technical effects of the present invention are not limited to the above-mentioned effects, and other technical effects not mentioned will be clearly understood by those skilled in the art from the following description.

### BRIEF DESCRIPTION OF THE DRAWINGS

FIG. 1 is a schematic diagram of a medical image processing system according to an embodiment of the present disclosure,

FIG. 2 is a conceptual diagram illustrating a diagnostic aid algorithm mounted on a medical image processing system according to an embodiment of the present disclosure,

FIG. 3 is a conceptual diagram illustrating an abnormal lesion classification model of a diagnostic aid algorithm according to the present embodiment,

FIG. 4 is a conceptual diagram illustrating a labeling method of an abnormal lesion classification model according to an embodiment of the present disclosure,

FIG. 5 is a conceptual diagram illustrating a learning method of an abnormal lesion classification model according to an embodiment,

FIG. 6 is a conceptual diagram illustrating a similarity evaluation method for extracting representative frames from a video frame processing model of a diagnostic aid algorithm according to an embodiment,

FIG. 7 is a conceptual diagram illustrating a concept of forcing extraction of representative frames from a video frame processing model of a diagnostic aid algorithm according to an embodiment,

FIG. 8 is a conceptual diagram illustrating an example of extracting representative frames from a video frame processing model of a diagnostic aid algorithm, according to an embodiment,

FIG. 9 is a conceptual diagram illustrating an abnormal lesion classification model of a diagnostic aid algorithm, according to another embodiment.

### DETAILED DESCRIPTION OF THE DISCLOSURE

Embodiments of the present disclosure will be described in detail below with reference to the accompanying drawings. However, the present disclosure is not limited to the embodiments disclosed herein and may be implemented in various forms, and these embodiments are provided only to make the disclosure complete and to give those of ordinary skill in the art a complete idea of the scope of the invention. The shapes of the elements in the drawings may be exaggerated for the sake of clarity, and elements designated by like numerals in the drawings are intended to be identical.

### Medical Image Processing System

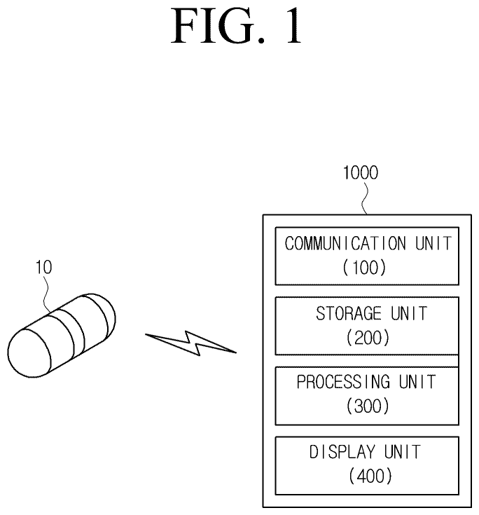

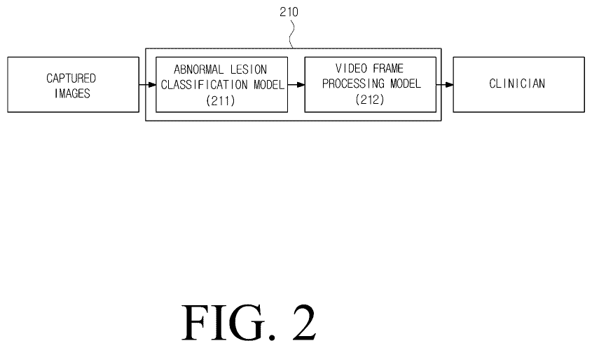

FIG. 1 is a schematic diagram of a medical image processing system according to the present embodiment, and FIG. 2 is a conceptual diagram of a diagnostic aid algorithm mounted on the medical image processing system according to the present embodiment.

As shown in FIGS. 1 and 2, the medical image processing system 1000 (hereinafter referred to as the processing system) according to the present embodiment receives captured images of the intestine provided by the capsule endoscope 10. The processing system 1000 selects lesion images including abnormal lesions from the captured images. The processing system 1000 then reduces the number of selected lesion images to generate an optimal readout image.

In one example, the processing system 1000 may include a communication unit 100, a storage unit 200, a processing unit 300, and a display unit 400.

The communication unit 100 receives captured images provided by the capsule endoscope 10. At this time, the communication unit 100 may receive the captured images via human body communication with the capsule endoscope 10. However, the communication method of the communication unit 100 can be implemented in various ways and is not limited to human body communication.

The storage unit 200 stores a diagnostic aid algorithm 210 that generates readout images based on the captured images. The diagnostic aid algorithm 210 may include an abnormal lesion classification model 211 and a video frame processing model 212. The abnormal lesion classification model 211 and the video frame processing model 212 may be deep a learning model that have been trained through prior learning.

The processing unit 300 generates readout images based on the captured images. The processing unit 300 applies the captured images to the abnormal lesion classification model 211 and the video frame processing model 212 in turn. Accordingly, the processing unit 300 generates readout images based on the diagnostic aid algorithm 210.

The display unit 400 may process the readout image and finally output the generated readout image. Accordingly, a clinician may perform a lesion diagnosis based on the readout image output from the display unit 400.

### Abnormal Lesion Classification Model Yolo v4

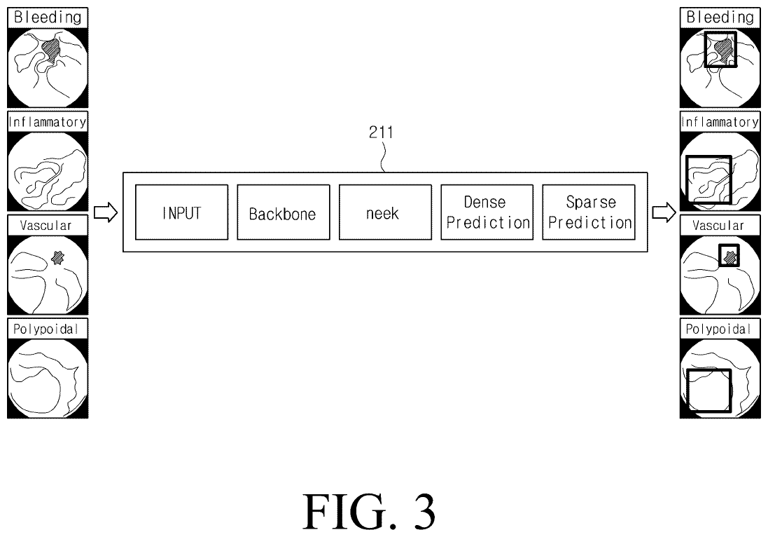

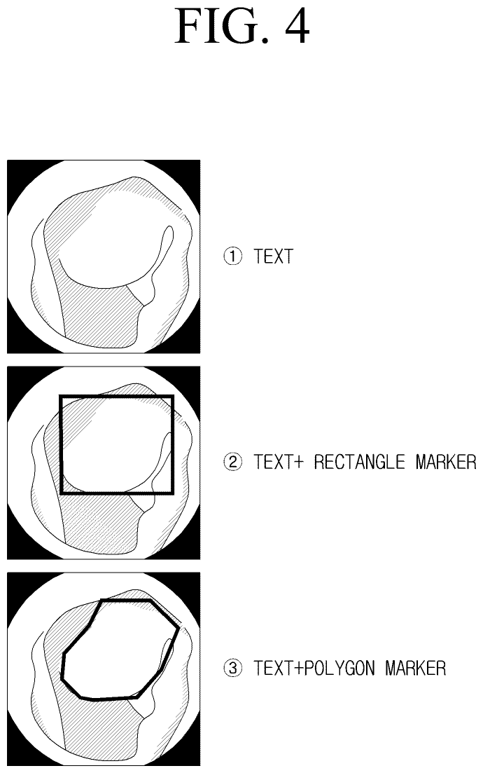

FIG. 3 is a conceptual diagram illustrating an abnormal lesion classification model of a diagnostic aid algorithm according to an embodiment, and FIG. 4 is a conceptual diagram illustrating a method for labeling an abnormal lesion classification model according to an embodiment.

As shown in FIGS. 3 and 4, the abnormal lesion classification model 211 according to the present embodiment binary classifies the captured images. That is, the abnormal lesion classification model 211 categorizes the captured images into lesion images and normal images. And the abnormal lesion classification model 211 can output the lesion image by applying a bounding box to a region where a lesion is located in the lesion image.

The abnormal lesion classification model 211 may apply various object detection models. For example, the abnormal lesion classification model 211 may be a YOLO (You Only Look Once) based model. Accordingly, labeling of image data applied to the abnormal lesion classification model 211 may involve various labeling methods depending on the model.

For example, in labeling the image data, an expert may identify a lesion present in the image data, and the expert may label text for the lesion.

In one example, in labeling the imaging data, an expert identifies a lesion present in the image data, and the expert labels text for the lesion and directly marks the lesion present in the image data. The expert may label the location of the lesion by drawing a rectangle around the lesion in the image data.

In one example, in labeling the imaging data, an expert identifies a lesion present in the image data, and the expert labels text for the lesion and directly marks the lesion present in the image data. The expert may label the location of the lesion by drawing a polygon around the lesion in the image data.

In the following, an embodiment will be described in which the abnormal lesion classification model 211 is arranged as a YOLO model to facilitate understanding of the invention. Labeling in such a YOLO model may involve an expert labeling text and drawing a rectangle around the lesion.

In one example, the YOLO model may be arranged as a YOLO v4 model. The YOLO v4 model adopts the YOLO v3, CSPDarknet53, SPP, PAN, BOF ALC BOS structure. However, the YOLO v4 model can show high performance in the detection of a small lesion by using a large input resolution.

Accordingly, the abnormal lesion classification model 211 to which the YOLO model is applied classifies images containing preset lesions when images are input, and performs a binary classification regardless of a type of a lesion to classify the images into lesion images and normal images. In this case, the preset lesions may include hemorrhagic, inflammatory, vascular, and polypoid lesions.

In general, when an image is input, the YOLO model can classify the image into four classes to determine whether the image contains hemorrhagic, inflammatory, vascular, and polypoid lesions. However, as the number of classes for classification increases, the performance of the deep learning model inevitably degrades.

Therefore, the abnormal lesion classification model 211 does not classify an image into four classes when the image is input. The abnormal lesion classification model 211 classifies an image containing a lesion regardless of the type of lesion, and classifies them into two classes: a lesion image and a normal image, known as ‘Binary Classification’. In other words, the abnormal lesion classification model 211 classifies an image containing at least one of hemorrhagic, inflammatory, vascular, and polypoid lesions, and reclassifies the classified image into a lesion image and a normal image. In this case, a criteria for distinguishing the lesion image from the normal image may be determined based on four thresholds after statistically calculating thresholds for each of the hemorrhagic, inflammatory, vascular, and polypoid lesions.

### Learning and Evaluation of the Abnormal Lesion Classification Model

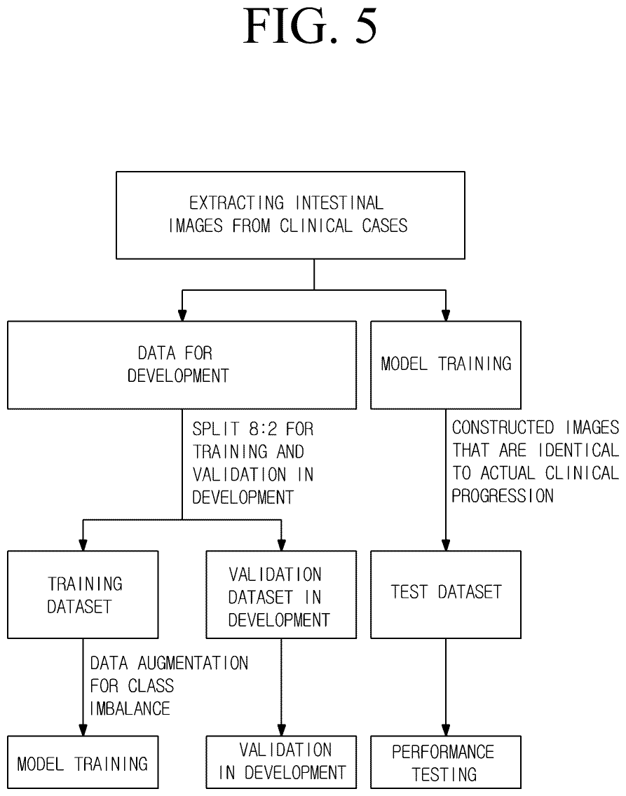

FIG. 5 is a conceptual diagram illustrating a learning method of an abnormal lesion classification model according to the present embodiment.

As shown in FIG. 5, in learning the abnormal lesion classification model 211 according to the present embodiment, model training can be performed, and validation and performance testing can be performed.

For example, in training the abnormal lesion classification model 211, 5,462,422 intestine images were extracted from clinical cases of 10,386 patients. The intestine images were categorized into development data and test data.

A total of 162,160 images were applied to the development data. Of these, 109,902 images are normal and 52,258 images are lesions. The lesion images consisted of 10,000 images of hemorrhagic lesions, 28,818 images of inflammatory lesions, 9,103 images of vascular lesions, and 4,337 images of polypoid lesions. The development data was divided into training data set and development validation data set. In this case, 80% of the development data was used as the training data set and 20% of the development data was used as the development validation data set.

The training dataset contains 87,922 normal images and 41,807 lesion images. The lesion images consisted of 8,000 images of hemorrhagic lesions, 23,055 images of inflammatory lesions, 7,282 images of vascular lesions, and 3,470 images of polyps. Thus, a total of 129,729 training data sets were used to train the abnormal lesion classification model.

The development validation dataset consists of 21,980 normal images and 10,451 lesion images. The lesion images consisted of 2,000 images of hemorrhagic lesions, 5,763 images of inflammatory lesions, 1,821 images of vascular lesions, and 867 images of polyps. Thus, a total of 32,431 developmental validation datasets were used to validate the training of the abnormal lesion classification model.

After training and validation, the abnormal lesion classification model 211 was subjected to performance testing.

A total of 5,300,262 images were applied to the test data. Of these, 4,829,022 images are normal images and 471,240 images are lesion images. The lesion images consisted of 471,240 images of hemorrhagic lesions, 150,237 images of inflammatory lesions, 17,242 images of vascular lesions, and 27,204 images of polyps. The test data was composed of images that were identical to actual clinical progression.

Then, In the performance evaluation and inference time test of the final trained abnormal lesion classification model 211, the abnormal lesion classification model 211 with the YOLO model performed well in binary classification of the imaging images.

### Video Frame Processing Model

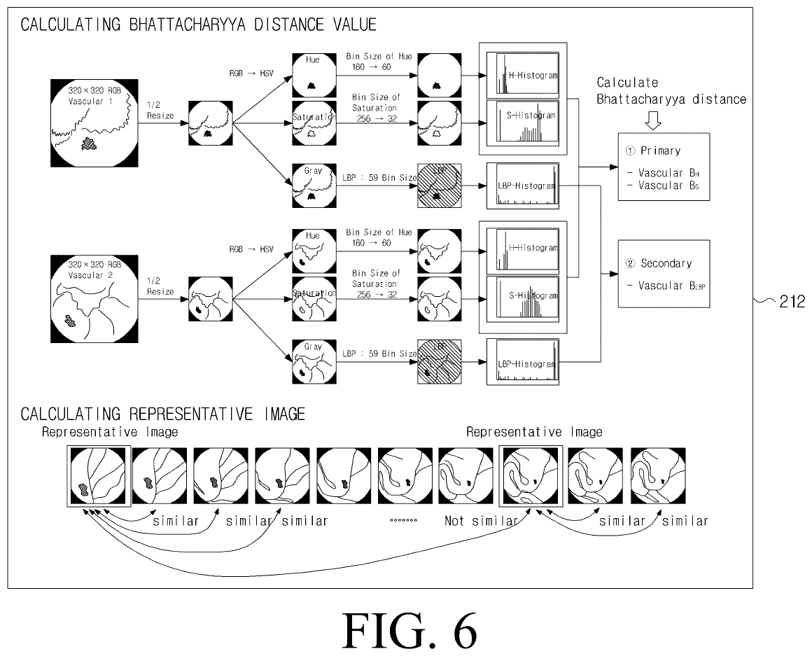

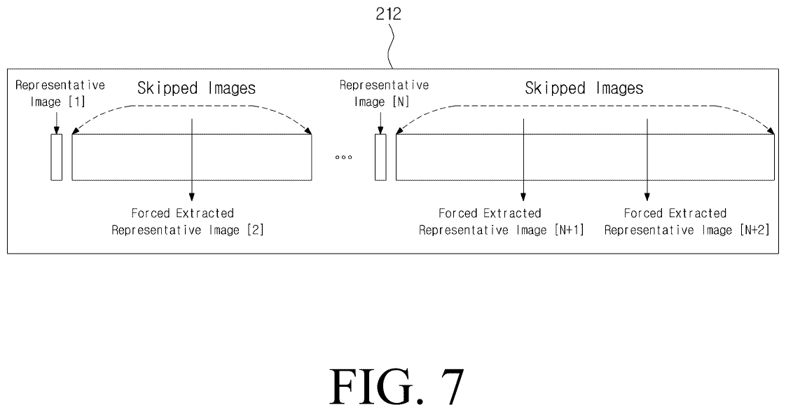

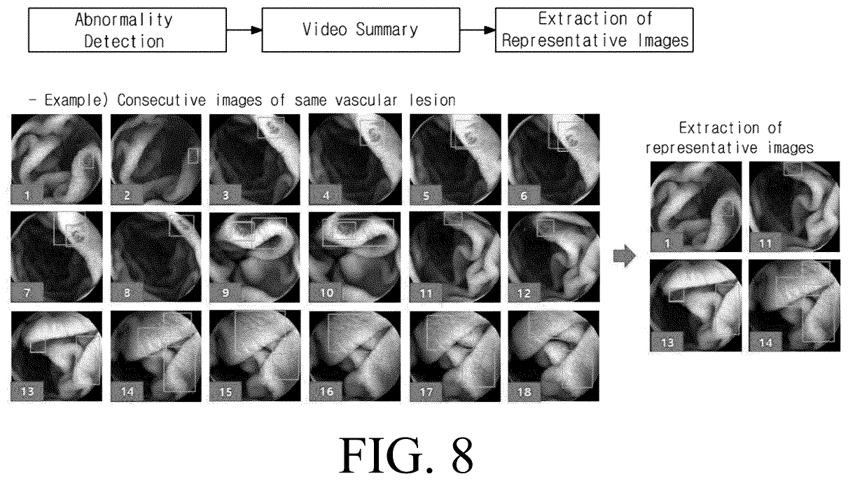

FIG. 6 is a conceptual diagram illustrating a similarity evaluation method for extracting a representative frame from a video frame processing model of a diagnostic aid algorithm according to the present embodiment, and FIG. 7 is a conceptual diagram illustrating a concept of forcing a representative frame from a video frame processing model of a diagnostic aid algorithm according to the present embodiment. FIG. 8 is a conceptual diagram illustrating an example of extracting representative frames from a video frame processing model of a diagnostic aid algorithm, according to an embodiment.

As shown in FIGS. 6 through 8, the video frame processing model 212 according to the present embodiment selects representative images, i.e., representative frames, of the lesion images provided from the abnormal lesion classification model 211. At this time, the selected representative frames may be output from the display unit 400.

The video frame processing model 212 compares the plurality of frames of the lesion images for similarity. The video frame processing model 212 then selects a representative frame that is representative of the similar frames. The video frame processing model 212 may extract features for color and texture of the frames to compare the similarity of the two frames.

The video frame processing model 212 then analyzes the similarity of the two frames using the Bhattacharyya Distance.

In one example, the value of the Bhattacharyya Distance may range from 0 to 1. In this case, the video frame processing model 212 may determine the similarity between the frames by determining that the value of the Bhattacharyya Distance is zero or closer to zero, such that the similarity is higher.

For example, it is assumed that the plurality of frames consist of frames 1 through 10, as shown in FIG. 6. Accordingly, the video frame processing model 212 uses the frame 1 as a reference frame and sequentially compares the reference frame to subsequent frames. In this case, from the frame 1 to the frame 7, the value of the Bhattacharyya Distance may be zero or close to zero. And the Bhattacharyya Distance of the frame 1 and the frame 8 may be 1 or close to 1.

Accordingly, the video frame processing model 212 determines that the frames 1 through 7 are similar frames. The video frame processing model 212 then selects a representative frame of the similar frames. Then, the video frame processing model 212 performs comparisons of a reference frame (=the frame 8) and subsequent frames in turn based on the frame 8, which is determined to be a different frame from the frame 1. The video frame processing model 212 then selects a representative frame for similar frames.

In the process of selecting the representative frame, the video frame processing model 212 may select the reference frame as the representative frame, that is, the frame that is the subject of the initial similarity comparison may be selected as the representative frame. For example, referring to FIG. 6, the frame 1 and the frame 8 may each be selected as representative frames.

Meanwhile, in the Bhattacharyya Distance calculation, the video frame processing model 212 performs normalization of the two frames to be compared. The video frame processing model 212 may then perform a histogram analysis to calculate the Bhattacharyya Distance.

As an example, the video frame processing model 212 will now describe calculating the Bhattacharyya Distance for frame 1 and frame 2. In this example, the frames may be RGB images of size 320×320. The video frame processing model 212 then resizes each image by ½. The video frame processing model 212 then performs an HSV conversion on the resized images, i.e., the video frame processing model converts the Red, Green, and Blue values to Hue, Saturation, and Value values.

The video frame processing model 212 then adjusts the number of bins of color and saturation values to perform a 2D histogram analysis. In this case, the video frame processing model 212 adjusts the number of bins of the color values from 180 to 60, and the video frame processing model 212 adjusts the saturation values from 256 to 32. The video frame processing model 212 then calculates a histogram (H-Histogram) for the color values and a histogram (S-Histogram) for the saturation values.

The video frame processing model 212 may then calculate a value of the Bhattacharyya Distance based on the histograms for the color values and the histograms for the saturation values for each of the first and second frames.

On the other hand, the video frame processing model 212 ensures that frames are not excessively skipped in selection of representative frame. Analyzing the similarity of frames through the value of the Bhattacharyya Distance may skip many frames that exist between the representative frames. However, in the medical field, if frames are skipped by simply analyzing similarity, unexpected problems may be found. Accordingly, the video frame processing model 212 may force extraction of representative frame by a predetermined frame interval N.

In one example, the video frame processing model 212 may select representative frames such that no more than 10 frames are selected. Referring to frames 1 through 18 of FIG. 8, from frame 1 through frame 12, the value via the Bhattacharyya Distance may be zero or close to zero. Then, starting from frame 13, the value of the Bhattacharyya Distance can be 1 or close to 1. And in the frame 14, the value of the Bhattacharyya Distance from the frame 13 may be 1 or close to 1. In this case, if the video frame processing model 212 utilizes only the value of the Bhattacharyya Distance, the frame 1, the frame 13, and the frame 14 are selected as representative frames.

However, as described above, the video frame processing model 212 may be set such that selection interval of the representative frames does not exceed 10 frames. Thus, even if the frame 1 to the frame 12 are determined to have a high similarity, the video frame processing model 212 selects the frame 1, the frame 11, the frame 13, and the frame 14 as representative frames. In other words, the video frame processing model 212 selects the frame 1 as an initial reference. The video frame processing model 212 then selects the frame 11, which is more than 10 frames from the frame 1, as a representative frame, and selects the frame 13, which has a different similarity to the frame 11, as a representative frame. The video frame processing model 212 may then select the frame 14 as a representative frame that has a different similarity to the frame 13.

The video frame processing model 212 then causes the selected representative frames to be output from the display unit 400. Thus, the clinician may use the representative frames to reduce the time required to diagnose the lesion.

### Performance Evaluation Result of the Medical Image Processing System

To evaluate the performance of the processing system 1000 according to the present invention, a performance evaluation was performed on an NVIDIA GeForce RTX 2080 and Window 10. The processing system 1000 can reduce a clinician's reading time from 30 minutes to over 2 hours to less than 10 minutes. In addition, the processing system 1000 showed a high performance in terms of sensitivity of 93.0%, specificity of 89.0%, and accuracy of 90.0%. In addition, the processing system 1000 showed a compression ratio of more than 80% of the number of video frames.

In the present embodiment, the abnormal lesion classification model is prepared with YOLO v4. However, in other embodiments, the abnormal lesion classification model may be configured to include YOLO v8.

### Abnormal Lesion Classification Model Yolo v8

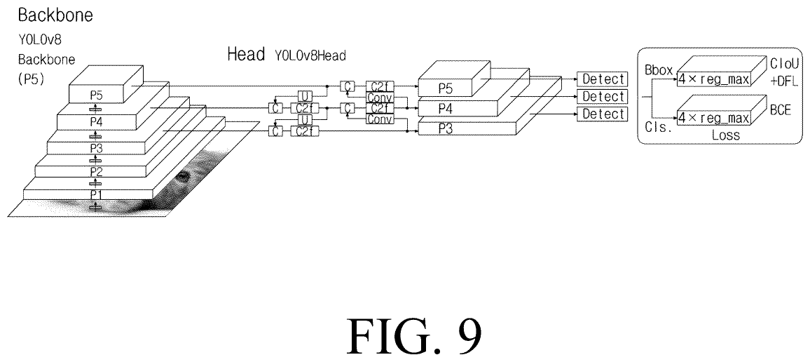

FIG. 9 is a conceptual diagram illustrating an abnormal lesion classification model of a diagnostic aid algorithm, according to another embodiment.

As shown in FIG. 9, the YOLO model according to the present embodiment may be arranged as a YOLO v8 model.

In one example, the YOLO v8 model utilizes a modified CSPDarknet53 backbone. In particular, the YOLO v8 model replaces the CSPLayer used in YOLO v5 with the C2f module. As a result, the YOLO v8 model has an advantage of accelerating computation speed by pooling image features into a fixed-size map with the Partial Pyramid Pooling Fast (SPPF) layer. At this time, each convolution of the YOLO v8 model applies BN (Batch Normalization) and SiLU Activation, and the head part is divided into Process Objectness, Classification, and Regression Tasks, and each task is prepared to perform each task separately.

In this way, the medical image processing system and processing method according to the present invention have the effect of reducing the reading time of medical images, improving the reading efficiency of clinicians, and shortening the waiting time of patients.

The embodiments of the present invention described above and illustrated in the drawings should not be construed as limiting the technical ideas of the present invention. The scope of protection of the present invention is limited only by the claims, and those having ordinary skill in the art will be able to make various improvements and modifications to the technical ideas of the present invention. Such improvements and modifications will therefore fall within the scope of protection of the invention as long as they are obvious to a person of ordinary skill in the art.

1. A medical imaging processing system, comprising:
a communication unit configured to receive images captured from a capsule endoscope; and
a processing unit configured to generate readout images from the captured images using lesion model results trained by a lesion classification method,
wherein the processing unit configured to apply the captured images to a pre-trained abnormal lesion classification model to classify images containing pre-set lesion, reclassify the classified images into lesion images and normal images, select representative images representing the lesion images from the classified lesion images, and generate the selected representative images as the readout images.

2. The medical imaging processing system of claim 1,
wherein the abnormal lesion classification model includes a YOLO (You Only Look Once) series model.

3. The medical imaging processing system of claim 1,
wherein the abnormal lesion classification model includes at least one of YOLO v4 and YOLO v8 models.

4. The medical imaging processing system of claim 3,
wherein, in labeling of training data for the abnormal lesion classification model, an expert identifies a lesion present in the training data, and performs labeling by applying the text for the lesion and a preset marking method to the lesion present in the training data.

5. The medical imaging processing system of claim 4,
wherein, in marking of the lesion, the lesion is marked with a rectangle or a polygon surrounding the lesion.

6. The medical imaging processing system of claim 1, wherein,
the captured images includes at least one of hemorrhagic, inflammatory, vascular, and polypoid lesion images; and
the abnormal lesion classification model binary classifies the captured images into the lesion images and the normal images regardless of a type of the lesion in the reclassification process.

7. The medical imaging processing system of claim 6,
wherein the abnormal lesion classification model applies a bounding box to a lesion included in the lesion image.

8. The medical imaging processing system of claim 6,
wherein the abnormal lesion classification model statistically calculates a threshold for each lesion comprising at least one of the hemorrhagic, inflammatory, vascular, and polypoid lesions, and
when the captured images is provided, binary classifies the lesion images and the normal images based on the threshold for each lesion.

9. The medical imaging processing system of claim 1,
wherein, in the selection of the representative images, a pre-trained video frame processing model is applied, and the video frame processing model extracts features of color and texture of the lesion images to compare the similarity between images.

10. The medical imaging processing system of claim 9,
wherein, in the similarity analysis, the similarity is analyzed based on Bhattacharyya Distance.

11. The medical imaging processing system of claim 9,
wherein, in the similarity analysis, the lesion images being compared with each other are HSV transformed, and histograms of color values and saturation values are calculated by adjusting the number of bins of color and saturation values.

12. The medical imaging processing system of claim 1,
wherein, in the selection of the representative images, comparisons between a reference image and subsequent images are sequentially performed, when a subsequent image with a different similarity from the reference image appears, a comparison between the reference image and the subsequent image is sequentially performed using the subsequent image with the different similarity as the reference image, and the reference images are selected as the representative images.

13. The medical imaging processing system of claim 12,
wherein, in the selection of the representative images, the representative images are selected so that the number of images between a pair of representative images does not exceed.

14. The medical imaging processing system of claim 1,
wherein, in the selection of the representative images, comparisons between a reference image and subsequent images are sequentially performed, a nth subsequent image after the reference image is selected as a reference image, and the reference image is selected as a representative image.

15. A method for processing medical images performed by a computing device, the method comprising the steps of:
receiving images captured from a capsule endoscope;
reclassifying the captured images into lesion images and normal images after applying the captured images to a pre-trained abnormal lesion classification model to classify images containing pre-set lesions;
selecting representative images that represent the lesion images from the classified lesion images; and
generating the selected representative images as readout images.
